(19) United States
(12) Patent Application Publication
(10) Pub. No.: US 2021/0315602 A1
(43) Pub. Date: Oct. 14, 2021

(54) TREATMENT TOOL

(71) Applicant: OLYMPUS CORPORATION, Tokyo (JP)

(72) Inventors: Ojiro KITAMURA, Tokyo (JP); Kazuhiro TANAKA, Tokyo (JP)

(73) Assignee: OLYMPUS CORPORATION, Tokyo (JP)

(21) Appl. No.: 17/358,716

(22) Filed: Jun. 25, 2021

Related U.S. Application Data
(63) Continuation of application No. PCT/JP2018/048542, filed on Dec. 28, 2018.

(51) Int. Cl. A61B 17/29

(52) U.S. Cl. CPC A61B 17/29; A61B 2018/00178

(57) ABSTRACT

A treatment tool includes: a sheath that extends along a first axis; an end effector that is arranged on the sheath, the end effector being configured to apply energy to living tissue according to power that is supplied; a first operation portion into which the sheath is inserted and that is rotatable on the first axis together with the sheath; a housing configured to support the first operation portion; a conductive wire that is laid at least in the housing and in the first operation portion, is electrically connected to the end effector, and serves as a path via which the power is supplied; and a rotation portion that is at least partially positioned in the housing, that is rotatable on the first axis together with the sheath and the first operation portion, and around which the conductive wire is wound in the housing.

### CROSS-REFERENCE TO RELATED APPLICATION

This application is a continuation of International Application No. PCT/JP2018/048542, filed on Dec. 28, 2018, the entire contents of which are incorporated herein by reference.

### BACKGROUND

### 1. Technical Field

The disclosure relates to a treatment tool.

### 2. Related Art

A treatment tool that treats a region to be treated (“subject region” below) in living tissue by applying energy to the subject region has been known (for example, refer to Japanese Patent No. 4350379).

The treatment tool (bipolar forceps) described in Japanese Patent No. 4350379 includes a shaft, an end effector (end effector assembly), a first operation portion (rotation assembly), a housing, and a conductive wire (a cable conductor). The shaft extends along a first axis. The end effector applies energy to living tissue according to supplied power. The shaft is inserted into the first operation portion and the first operation portion is rotatable about the first axis together with the shaft according to an operation of a user. The housing supports the first operation portion such that the first operation portion is rotatable about the first axis. The conductive wire is laid in the housing, in the first operation portion, and in the shaft, is electrically connected to the end effector, and serves as a path via which the aforementioned power is supplied.

The treatment tool described in Japanese Patent No. 4350379 employs a configuration in which the conductive wire is wound in the first operation portion in order to prevent the conductive wire from breaking when an excessive tension occurs to the conductive wire when the shaft and the first operation portion rotate according to an operation of the user.

### SUMMARY

In some embodiments, a treatment tool includes: a sheath that extends along a first axis; an end effector that is arranged on the sheath, the end effector being configured to apply energy to living tissue according to power that is supplied; a first operation portion into which the sheath is inserted and that is rotatable on the first axis together with the sheath according to an operation of a user; a housing configured to support the first operation portion such that the first operation portion is rotatable on the first axis; a conductive wire that is laid at least in the housing and in the first operation portion, is electrically connected to the end effector, and serves as a path via which the power is supplied; and a rotation portion that is at least partially positioned in the housing, that is rotatable on the first axis together with the sheath and the first operation portion, and around which the conductive wire is wound in the housing.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of a treatment tool according to an exemplary embodiment;

FIG. 2 is a diagram illustrating the configuration of the treatment tool according to the embodiment;

FIG. 3 is a diagram illustrating the configuration of the treatment tool according to the embodiment;

FIG. 4 is a diagram illustrating a wiring path of a conductive wire; and

FIG. 5 is a diagram illustrating the wiring path of the conductive wire.

### DETAILED DESCRIPTION

With reference to the accompanying drawings, a mode for carrying out the disclosure (“embodiment” below) will be described below. The embodiment described below does not limit the disclosure. Furthermore, in the illustration of the drawings, the same components are denoted with the same reference number.

Configuration of Treatment Tool

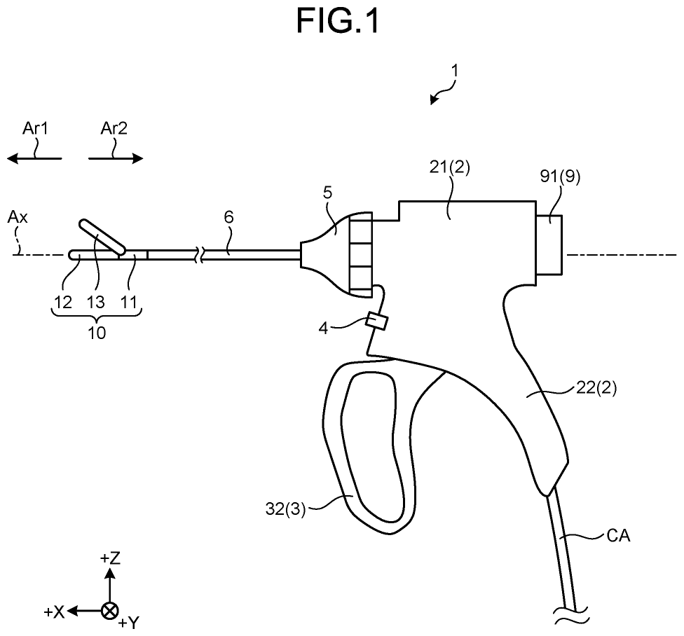

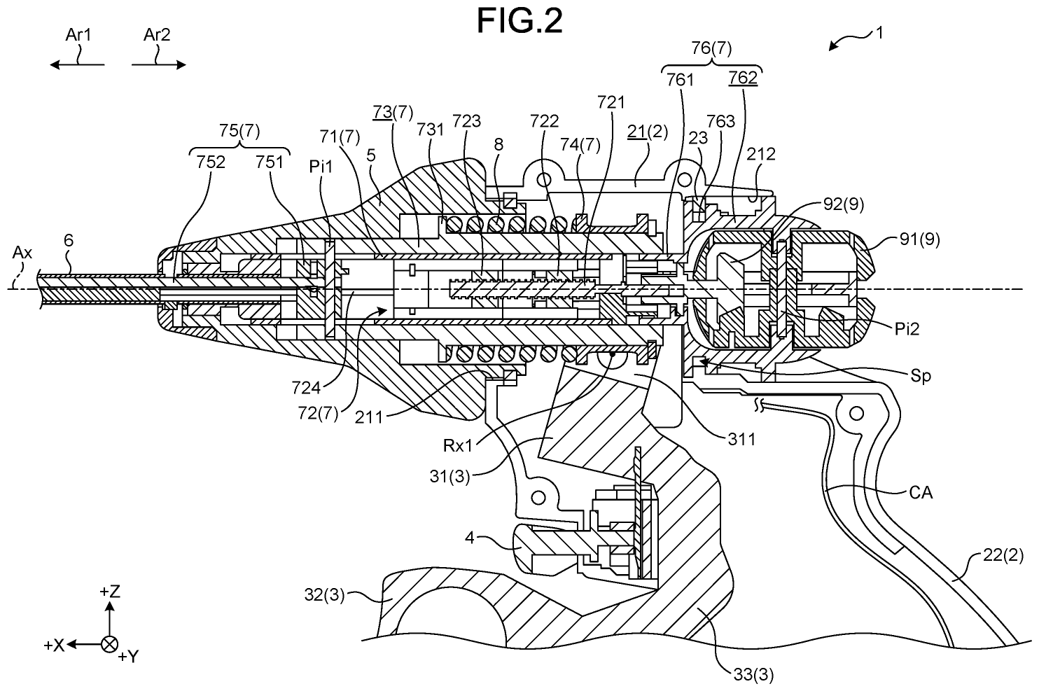

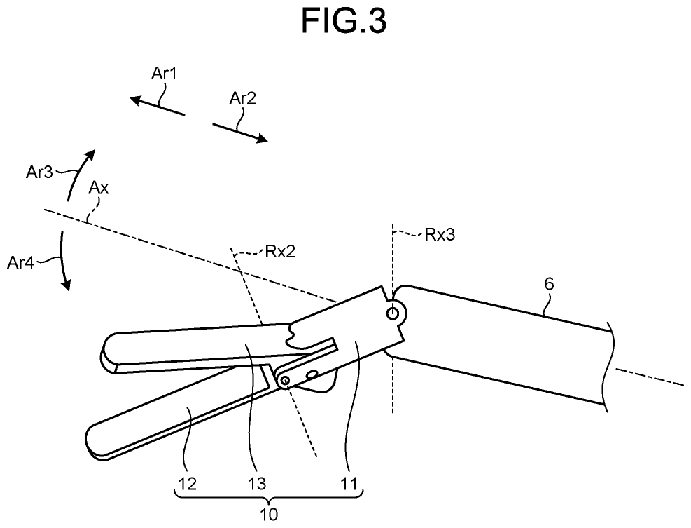

FIGS. 1 to 3 are diagrams illustrating a configuration of a treatment tool 1 according to the embodiment. Specifically, FIG. 1 is a diagram illustrating an overall configuration of the treatment tool 1. FIG. 2 is a diagram illustrating an internal configuration of a housing 2. FIG. 3 is a diagram illustrating a distal end part of the treatment tool 1.

Note that, in FIGS. 1 and 2, X, Y and Z coordinate axes consisting of an X-axis, a Y-axis and a Z-axis orthogonal to one another are used. The X-axis is an axis parallel to a center axis Ax (FIG. 1 and FIG. 2) of a sheath 6. The center axis Ax corresponds to a first axis according to the disclosure. The Y-axis is an axis orthogonal to the drawing planes of FIG. 1 and FIG. 2. The Z-axis is an axis along the top-bottom direction in FIG. 1 and FIG. 2. One side (+X-axis side) along the center axis Ax is referred to as a distal end side Ar1 and the other side (−X-axis side) is referred to as a proximal end side Ar2.

The treatment tool 1 treats a region to be treated (“subject region” below) in living tissue by applying treatment energy to the subject region. The treating herein refers to, for example, coagulating and cutting the subject region. As illustrated in FIGS. 1 to 3, the treatment tool 1 includes a housing 2 (FIG. 1 and FIG. 2), a movable handle 3 (FIG. 1 and FIG. 2), a switch 4 (FIG. 1 and FIG. 2), a rotation knob 5 (FIG. 1 and FIG. 2), the sheath 6, a rotation portion 7 (FIG. 2), a coil spring 8 (FIG. 2), a flex operation portion 9 (FIG. 1 and FIG. 2), an end effector 10 (FIG. 1 and FIG. 3), and a conductive wire CA (FIG. 1 and FIG. 2).

The housing 2 supports the entire treatment tool 1. As illustrated in FIG. 1 or FIG. 2, the housing 2 includes a housing body 21 that has an approximately cylindrical shape coaxial with the center axis Ax and a fixed handle 22 that extends from the housing body 21 to the −Z-axis side (the bottom side in FIG. 1 and FIG. 2) and that is held by an operator, such as a practitioner.

The movable handle 3 receives a closing operation and an opening operation of the operator, such as a practitioner. As illustrated in FIG. 2, the movable handle 3 includes a handle base 31, a handle body 32, and a handle joint 33.

The handle base 31 is positioned in the housing 2. A part of the handle base 31 on the +Z-axis side (top side in FIG. 2) is supported axially on the housing 2 such that the part is rotatable about a first rotation axis Rx1 (FIG. 2) parallel to the Y-axis. In an end of the handle base 31 on the +Z-axis side, engaging parts 311 (FIG. 2) in a pair facing each other along the Y-axis direction are arranged with a slider 74 (FIG. 2) interposed in between, which is a slider that forks and protrudes to the +Z-axis side and configures the rotation portion 7. The engaging parts 311 in a pair are parts that are engaged with the slider 74. Note that FIG. 2 illustrates only the engaging part 311 in the +Y-axis direction (depth direction with respect to the drawing plane of FIG. 2) out of the engaging parts 311 in a pair.

The handle body 32 is a part that receives the opening operation and the closing operation of the operator, such as a practitioner, and, as illustrated in FIG. 1 or FIG. 2, the handle body 32 is positioned outside the housing 2.

As illustrated in FIG. 2, the handle joint 33 is a part that is arranged, straddling the inside and outside the housing 2, and that connects the handle base 31 and the handle body 32.

When the closing operation of the operator, such as a practitioner, is received, the movable handle 3 rotates counterclockwise on the first rotation axis Rx1 in FIG. 2. On the other hand, when the opening operation of the operator, such as a practitioner, is received, the movable handle 3 rotates clockwise on the first rotation axis Rx1 in FIG. 2.

As illustrated in FIG. 1 or FIG. 2, the switch 4 is arranged in a state of being exposed to the outside from a side surface of the fixed handle 22 on the distal end side Ar1. The switch 4 receives an output start operation of the operator, such as a practitioner. The output start operation is an operation of starting application of treatment energy to the subject region.

The rotation knob 5 corresponds to a first operation portion according to the disclosure. The rotation knob 5 has an approximately cylindrical shape extending along the center axis Ax and is arranged in a posture such that the rotation knob 5 is coaxial with the center axis Ax. More specifically, the rotation knob 5 is supported by the housing body 21 rotatably on the center axis Ax with its end on the proximal end side Ar2 being inserted into a distal end opening 211 (FIG. 2) of the housing body 21 on the distal end side Ar1. The rotation knob 5 receives a rotation operation of the operator, such as a practitioner. According to the rotation operation, the rotation knob 5 rotates on the center axis Ax with respect the housing body 21.

The sheath 6 has an approximately cylindrical shape. As illustrated in FIG. 3, the end effector 10 is arranged on an end of the sheath 6 on the distal end side Ar1. An end of the sheath 6 on the proximal end side Ar2 is inserted into the rotation knob 5 and is fixed to an inner surface of the rotation knob 5 by welding, or the like. In other words, the sheath 6 and the end effector 10 rotate on the center axis Ax together with the rotation knob 5 according to the rotation operation of the operator, such as a practitioner, on the rotation knob 5.

According to the rotation operation of the operator, such as a practitioner, on the rotation knob 5, the rotation portion 7 rotates on the center axis Ax together with the rotation knob 5. As illustrated in FIG. 2, the rotation portion 7 includes a first holder member 71, a drive mechanism 72, a slider receiver 73, the slider 74, an open-close mechanism 75, and a second holder member 76.

The first holder member 71 corresponds to a second rotation part according to the disclosure. As illustrated in FIG. 2, the first holder member 71 has an approximately cylindrical shape extending along the center axis Ax and is arranged in a posture such that the first holder member 71 is coaxial with the center axis Ax. More specifically, the first holder member 71 is inserted into the rotation knob 5 and the housing body 21, straddling the rotation knob 5 and the housing body 21. An end of the first holder member 71 on the distal end side Ar1 is fixed to the inner surface of the rotation knob 5 by welding, or the like. In other words, the first holder member 71 rotates on the center axis Ax together with the rotation knob 5 according to the rotation operation of the operator, such as a practitioner, on the rotation knob 5.

The first holder member 71 described above internally holds each of part of the drive mechanism 72 and part of the open-close mechanism 75.

The configuration of the drive mechanism 72 will be described together with the configuration of the flex operation portion 9.

The slider receiver 73 corresponds to a first rotation part according to the disclosure. As illustrated in FIG. 2, the slider receiver 73 has a cylindrical shape extending along the center axis Ax and is arranged in a posture such that the slider receiver 73 is coaxial with the center axis Ax. More specifically, in a state where the slider receiver 73 is inserted into the coil spring 8 and the first holder member 71 is inserted into the slider receiver 73, the slider receiver 73 is arranged movably with respect to the first holder member 71 along the center axis Ax. An end of the slider receiver 73 on the distal end side Ar1 is fixed to the open-close mechanism 75 that is held in the first holder member 71 with a first pin Pi1 (FIG. 2) in a state where move of the end of the slider receiver 73 along the center axis Ax with respect to the first holder member 71 is allowed and rotation on the center axis Ax is restricted. In other words, the slider receiver 73 rotates on the center axis Ax together with the rotation knob 5 and the first holder member 71 according to the rotation operation of the operator, such as a practitioner, on the rotation knob 5.

As illustrated in FIG. 2, in the slider receiver 73, a jutting part 731 that protrudes from an outer circumferential surface and that extends over the circumference in a circumferential direction around the center axis Ax is formed.

As illustrated in FIG. 2, the slider 74 has an approximately cylindrical shape and is arranged in a posture such that the slider 74 is coaxial with the center axis Ax. More specifically, the slider 74 is arranged movably with respect to the slider receiver 73 along the center axis Ax with the slider receiver 73 being inserted into the slider 74. As described above, the slider 74 is engaged with the movable handle 3 by the engaging parts 311 in a pair.

The coil spring 8 has a function of applying a drive force to a second gripper 13 from a first gripper 12 and the second gripper 13 (FIG. 1 and FIG. 3) configuring the end effector 10 according to the close operation and the open operation of the operator, such as a practitioner, on the movable handle 3. The drive force is a drive force for opening and closing the second gripper 13 with respect to the first gripper 12. In other words, the coil spring 8 corresponds to an elastic material according to the disclosure. As illustrated in FIG. 2, the coil spring 8 is arranged in a state of being interposed between the jutting part 731 and the slider 74, with the slider receiver 73 being inserted into the coil spring 8.

The elastic material according to the disclosure is not limited to the coil spring 8 as long as the elastic material has a ring form around the center axis Ax and has the above-described function and a disc spring, or the like, may be used.

The open-close mechanism 75 is a mechanism that opens and closes the second gripper 13 with respect to the first gripper 12. As illustrated in FIG. 2, the open-close mechanism 75 includes an open-close joint 751 and a transmission shaft 752.

The open-close joint 751 is a part that is fixed to the slider receiver 73 with the first pin Pi1 and is held in the first holder member 71 movably along the center axis Ax.

The transmission shaft 752 is an elongated member that extends along the center axis Ax and is inserted into the sheath 6. An end of the transmission shaft 752 on the proximal end side Ar2 protrudes to the outside of the sheath 6 and is inserted into the first holder member 71 and is fixed to the open-close joint 751. In other words, the transmission shaft 752 is movable along the center axis Ax together with the open-close joint 751. An end of the transmission shaft 752 on the distal end side Ar1 is mechanically connected to the second gripper 13.

The slider 74, the slider receiver 73, and the open-close mechanism 75 move as described below according to an operation of the operator, such as a practitioner, on the movable handle 3.

According to the close operation of the operator, such as a practitioner, on the movable handle 3, the slider 74 is pushed into along the center axis Ax to the distal end side Ar1 by the engaging parts 311 in a pair. The slider receiver 73 receives a push force (drive force for opening and closing the second gripper 13 with respect to the first gripper 12) from the slider 74 to the distal end side Ar1 via the coil spring 8. The open-close mechanism 75 moves in association with the slider receiver 73 to the distal end side Ar1 along the center axis Ax. The open-close mechanism 75 applies (transmits) the drive force to the second gripper 13. Accordingly, the second gripper 13 rotates on a second rotation axis Rx2 (FIG. 3) in a direction in which the second gripper 13 moves close to the first gripper 12 (close direction).

On the other hand, when the open operation of the operator, such as a practitioner, on the movable handle 3 is performed, the slider 74, the slider receiver 73, and the open-close mechanism 75 move in a direction inverse to the above-described direction. Accordingly, the second gripper 13 rotates on the second rotation axis Rx2 in a direction in which the second gripper 13 separates from the first gripper 12 (open direction).

As described above, the second gripper 13 opens and closes with respect to the first gripper 12 according to an operation of the operator, such as a practitioner, on the movable handle 3.

The second holder member 76 is a member that holds the flex operation portion 9. As illustrated in FIG. 2, the second holder member 76 includes a fitting part 761 and a holder member body 762.

As illustrated in FIG. 2, the fitting part 761 is formed into a cylindrical shape having an outer diameter approximately equal to an inner diameter of the slider receiver 73 and is fitted into the slider receiver 73, thereby being connected to the slider receiver 73. In other words, the second holder member 76 rotates on the center axis Ax together with the rotation knob 5 and the slider receiver 73 according to the rotation operation of the operator, such as a practitioner, on the rotation knob 5.

As illustrated in FIG. 2, the holder member body 762 is formed in an approximately cylindrical shape having an outer diameter larger than an outer diameter of the slider receiver 73 and is formed integrally with the fitting part 761 and in a posture such that the holder member body 762 is coaxial with the fitting part 761. The holder member body 762 internally holds the flex operation portion 9 and is exposed to the outside of the housing body 21 from a proximal end opening 212 (FIG. 2) of the housing body 21 on the proximal end side Ar2.

In other words, the second holder member 76 is positioned at a proximal end of the rotation portion 7 and is connected to the slider receiver 73, thus corresponding to a third rotation part according to the disclosure. The second holder member 76 corresponds to a proximal end part of a rotation portion according to the disclosure.

The flex operation portion 9 corresponds to a second operation portion according to the disclosure. As illustrated in FIG. 2, the flex operation portion 9 includes a flex operation portion body 91 and a rotation converter 92.

As illustrated in FIG. 2, the flex operation portion body 91 has an overall approximately cylindrical shape. A cylindrical second pin Pi2 is inserted on the center axis of the flex operation portion body 91. With the second pin Pi2, the flex operation portion body 91 is held in the holder member body 762 rotatably on the second pin Pi2. In that state, the flex operation portion body 91 is positioned on the center axis Ax. The flex operation portion body 91 receives a flex operation of the operator, such as a practitioner. According to the flex operation, the flex operation portion body 91 rotates on the second pin Pi2 with respect to the holder member body 762.

The rotation converter 92 is connected to each of the flex operation portion body 91 and the drive mechanism 72. The rotation converter 92 converts the rotation on the second pin Pi2 according to a flex operation of the operator, such as a practitioner, on the flex operation portion body 91 into rotation on the center axis Ax. In other words, the rotation converter 92 rotates on the center axis Ax according to the flex operation. A bevel gear, or the like, can be exemplified as the rotation converter 92.

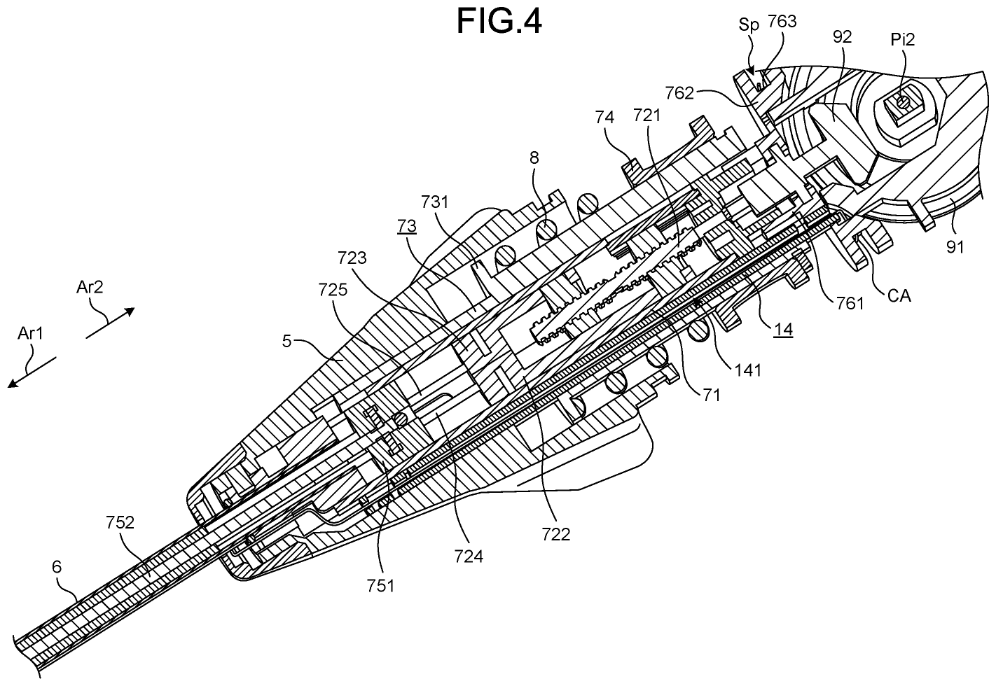

The drive mechanism 72 is a mechanism that causes the end effector 10 to flex with respect to the sheath 6. As illustrated in FIG. 2, the drive mechanism 72 includes a rotation shaft 721, first and second drivers 722 and 723, and first and second rods 724 and 725 (see FIG. 4).

The rotation shaft 721 is a cylindrical elongated member that extends along the center axis Ax and is inserted into the first holder member 71 and in a posture such that the rotation shaft 721 is coaxial with the center axis Ax. An end of the rotation shaft 721 on the proximal end side Ar2 is fixed to the rotation converter 92. In other words, the rotation shaft 721 rotates on the center axis Ax together with the rotation converter 92 according to the flex operation of the operator, such as a practitioner, on the flex operation portion body 91.

Each of the first and second drivers 722 and 723 is connected to the rotation shaft 721. The first and second drivers 722 and 723 move in association with rotation of the rotation shaft 721 on the center axis Ax and are held in the first holder member 71 movably in inverse directions along the center axis Ax. A screw structure consisting of left-hand screws can be exemplified as a structure that connects the first and second drivers 722 and 723 and the rotation shaft 721.

The first and second rods 724 and 725 are elongated members each of which extends along the center axis Ax and is inserted into the sheath 6. An end of the first rod 724 on the proximal end side Ar2 protrudes to the outside of the sheath 6, is inserted into the first holder member 71, and is fixed to the first driver 722. On the other hand, an end of the second rod 725 on the proximal end side Ar2 protrudes to the outside of the sheath 6, is inserted into the first holder member 71, and is fixed to the second driver 723. In other words, the first and second rods 724 and 725 are movable along the center axis Ax together with the first and second drivers 722 and 723. Each of ends of the first and second rods 724 and 725 on the distal end side Ar1 is mechanically connected to an end effector base (FIG. 1 and FIG. 3) forming the end effector 10.

The drive mechanism 72 moves as described below according to the flex operation of the operator, such as a practitioner, on the flex operation portion body 91.

When the operator, such as a practitioner, causes the flex operation portion body 91 to rotate about the second pin Pi2 in a first direction (flex operation), the operation force is transmitted from the flex operation portion body 91 to the first and second rods 724 and 725 via the rotation converter 92, the rotation shaft 721, and the first and second drivers 722 and 723. The first rod 724 moves along the center axis Ax to the distal end side Ar1 and pushes the end effector 10 to the distal end side Ar1. On the other hand, the second rod 725 moves along the center axis Ax to the proximal end side Ar2 and pulls the end effector base 11 to the proximal end side Ar2. Accordingly, the end effector 10 rotates on a third rotation axis Rx3 with respect to the sheath 6 in a first flex direction Ar3 (FIG. 3).

On the other hand, when the operator, such as a practitioner, causes the flex operation portion body 91 to rotate about the second pin Pi2 in a second direction that is a direction opposite to the above-described first direction (performs the flex operation), the first and second rods 724 and 725 moves in directions opposite to those described above, respectively. Accordingly, the end effector 10 rotates on the third rotation axis Rx3 with respect to the sheath 6 in a second flex direction Ar4 (FIG. 3) that is a direction opposite to the first flex direction Ar3 (FIG. 3).

As illustrated in FIG. 1 or FIG. 3, the end effector 10 includes the end effector base 11 and the first and second grippers 12 and 13.

The end effector base 11 has a tubular shape. One end side of the tubular shape (an end on the proximal end side Ar2) of the end effector base 11 is pivotally supported on the end of the sheath 6 on the distal end side Ar1 rotatably on the second rotation axis Rx2.

The first gripper 12 has an elongated shape that is an extension of part of the end effector base 11 to the distal end side. On the other hand, the second gripper 13 has an elongated shape like the first gripper 12 and one end of the second gripper 13 on the proximal end side Ar2 is pivotally supported on the end effector base 11 rotatably on the third rotation axis Rx3. As described above, the second gripper 13 opens and closes with respect to the first gripper 12 according to an operation of the operator, such as a practitioner, on the movable handle 3 and is able to grip the subject region with respect to the first gripper 12.

In a state of gripping the subject region between the first and second grippers 12 and 13, the end effector 10 applies treatment energy to the subject region according to supplied power, thereby treating the subject region. The power is supplied to the end effector 10 via the conductive wire CA according to the output start operation of the operator, such as a practitioner, on the switch 4.

Although not illustrated in the drawings, a heater or electrodes in a pair described below can be exemplified as a configuration that causes the treatment energy according to the supplied power.

The heater is a sheet heater that generates heat according to supplied power and is arranged in at least one of the first and second grippers 12 and 13. The heater is, for example, obtained by forming an electrical resistance pattern that generates heat by conduction by vapor deposition, or the like, on a sheet-like substrate made of an insulating material, such as polyimide. The end effector transmits heat from the heater to the subject region that is gripped between the first and second grippers 12 and 13, thereby treating the subject region.

As described above, in the configuration using the heater, thermal energy is used as the treatment energy that is applied to the subject region.

One of the electrodes in a pair is arranged on the first gripper 12. The other electrode is arranged on the second gripper 13. When power (high-frequency power) is supplied to the electrodes in a pair, a high-frequency current flows into the subject region that is gripped between the first and second grippers 12 and 13 so that joule heat is generated. Accordingly, the subject region is treated.

As described above, in the configuration using the electrodes in a pair, high-frequency energy is used as the treatment energy that is applied to the subject region.

The conductive wire CA is electrically connected to an external control device (not illustrated in the drawing) and is drawn into the housing 2 from an end of the fixed handle 22 on the −Z-axis side. The conductive wire CA is drawn from the end of the fixed handle 22 on the −z-axis side into the sheath 6 via the housing 2 and the rotation knob 5 and is electrically connected to the end effector 10 (the above-described heater, the electrodes in a pair, etc.). In other words, the conductive wire CA serves as a path via which power to be supplied is supplied from the external control device (not illustrated in the drawing) to the end effector 10 (the above-described heater, the electrodes in a pair, etc.).

The wiring path of the conductive wire CA in the housing 2 and the rotation knob 5 will be described below.

Wiring Path of Cable

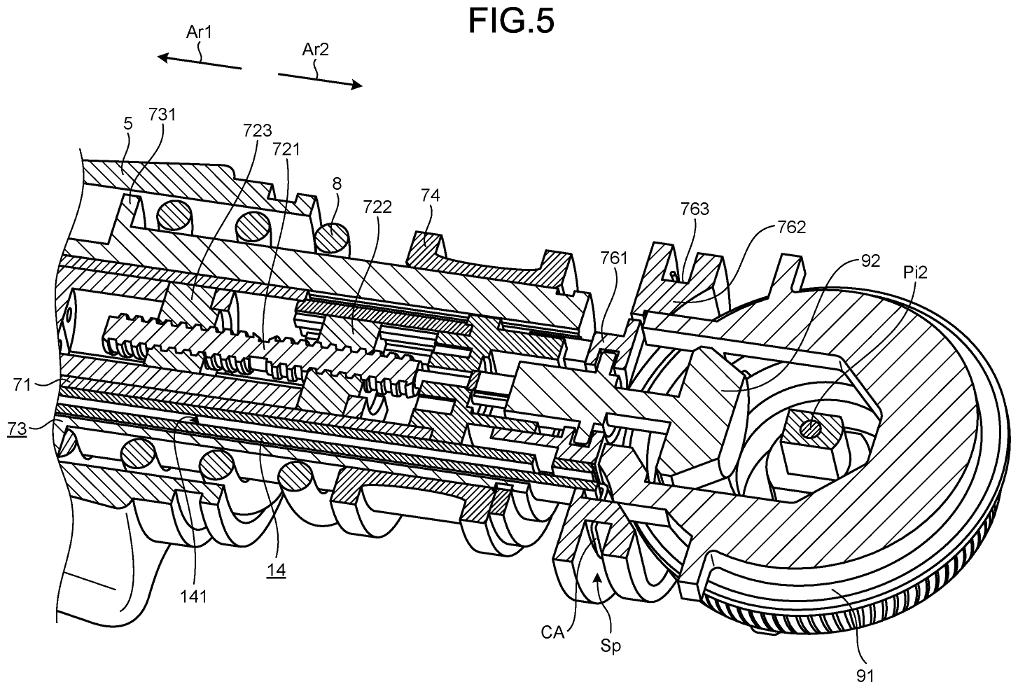

FIGS. 4 and 5 are diagrams illustrating the wiring path of the conductive wire CA.

For the purpose of illustration, FIG. 2 illustrates only the wiring path of the conductive wire CA from the end of the fixed handle 22 on the −Z-axis side to a position close to the second holder member 76. FIGS. 4 and 5 illustrate the wiring path from the position close to the second holder member 76 to the inside of the sheath 6.

As illustrated in FIG. 2, FIG. 4 or FIG. 5, an annular concave groove 763 that extends over the circumference in the circumferential direction around the center axis Ax is formed. In the housing 2, as illustrated in FIG. 2, a protrusion 23 that extends along the circumferential direction around the center axis Ax and that is inserted into the concave groove 763 is formed. In the state where the protrusion 23 is inserted into the concave groove 763, a specific space Sp (FIG. 2, FIG. 4 and FIG. 5) is formed in the concave groove 763.

Between the first holder member 71 and the slider receiver 73, as illustrated in FIG. 4 or FIG. 5, a path setting member 14 having a through-hole 141 that penetrates from an end face on the proximal end side Ar2 to an end face on the distal end side Ar1 and in which the conductive wire CA is laid is arranged. An end of the path setting member 14 on the proximal end side Ar2 is inserted into the holder member body 762.

The conductive wire CA that is laid to the position close to the second holder member 76 is wound in the space Sp as illustrated in FIG. 4 or FIG. 5. The wound conductive wire CA is inserted into the holder member body 762 via a through-hole (not illustrated in the drawing) penetrating from the bottom surface of the concave groove 763 into the holder member body 762. Furthermore, as illustrated in FIG. 4, the conductive wire CA inserted into the holder member body 762 is inserted into the through-hole 141 from the end face of the path setting member 14 on the proximal end side Ar2 and is drawn to the end face of the path setting member 14 on the distal end side Ar1 via the through-hole 141. The conductive wire CA that is drawn to the end face of the path setting member 14 on the distal end side Ar1 is inserted into the sheath 6 via a through hole (not illustrated in the drawing) that penetrates from the inside to the outside of the sheath 6 and is electrically connected to the end effector 10 (the heater and the electrodes in a pair described above, etc.).

According to the above-described embodiment, the following effects are achieved.

In the treatment tool 1 according to the embodiment, the conductive wire CA is wound around the rotation portion 7 in the housing 2. Thus, when the sheath 6, etc., rotate on the center axis Ax together with the rotation knob 5 according to an operation of the operator, such as a practitioner, on the rotation knob 5, the part of the conductive wire CA that is wound around the rotation portion 7 makes a first move of changing the state in a direction to loosen the part or makes a second move of returning to the original state again. In other words, the first and second moves make it possible to prevent occurrence of an excessive tension to the conductive wire CA and inhibit the conductive wire CA from breaking.

Particularly, the conductive wire CA is wound around the rotation portion 7 that rotates on the center axis Ax together with the rotation knob 5. Thus, the wound part rotates on the center axis Ax together with the rotation portion 7 and thus it is possible to inhibit the part from sliding on the outer circumferential surface on the rotation portion 7. Thus, compared with the configuration in which the conductive wire CA is wound around a member that does not rotate on the center axis Ax together with the rotation knob 5, it is possible to further inhibit the conductive wire CA from breaking.

The conductive wire CA is wound around the rotation portion 7 not in the rotation knob 5 but in the housing 2. Thus, it is unnecessary to additionally make a wiring space for the conductive wire CA in the rotation knob 5 and thus it is possible to reduce the size of the rotation knob 5. Accordingly, it is possible to realize a configuration in which even an operator with small hands, such as a female surgeon, easily operates the rotation knob 5 and improve operability.

In the treatment tool 1 according to the embodiment, the rotation portion 7 (the first holder member 71 and the slider receiver 73) is inserted into the coil spring 8. Furthermore, the conductive wire CA passes through the through-hole 141 in the path setting member 14 that is arranged between the first holder member 71 and the slider receiver 73 that configure the rotation portion 7. The drive mechanism 72 is arranged in the first holder member 71.

Thus, it is possible to collectively arrange the first holder member 71, the drive mechanism 72, the slider receiver 73 and the conductive wire CA in the annular shape of the coil spring 8 and thus it is unnecessary to make a space for arranging the members 71 to 73 and CA outside the annular shape of the coil spring 8. In other words, it is possible to reduce the size of the housing 2. Accordingly, it is possible to realize a configuration in which even an operator with small hands, such as a female surgeon, easily holds the treatment tool and further improve operability.

Particularly, having the conductive wire CA pass between the first holder member 71 and the slider receiver 73 makes it possible to prevent the conductive wire CA from mechanically interfering with the engaging parts 311 in a pair of the movable handle 3. In other words, preventing mechanical interference between the conductive wire CA and the engaging parts 311 in a pair makes it possible to further inhibit the conductive wire CA from breaking.

In the treatment tool 1 according to the embodiment, the conductive wire CA is wound in the space Sp that is formed by the concave groove 763 that is formed in the rotation portion 7 and the protrusion 23 that is formed in the housing 2.

This makes is possible to maintain the wound part in the space Sp and prevent the wound part from getting stacked in surrounding members when the first and second moves are made. Accordingly, it is possible to further inhibit the conductive wire CA from breaking.

Furthermore, in the treatment tool 1 according to the embodiment, the flex operation portion 9 that receives the flex operation of the user, such as a doctor, is arranged on the end on the center axis Ax and on the proximal end side Ar2. In other words, the treatment tool 1 according to the embodiment has a configuration in which the flex operation portion 9 is arranged in the aforementioned position and therefore it is not possible to draw the conductive wire CA from the position. In the configuration, it is necessary to draw the conductive wire CA from a position separate from the center axis Ax (the end of the fixed handle 22 on the −Z-axis side in the embodiment) and thus a tension tends to occur to the conductive wire CA according to an operation of the operator, such as a practitioner, on the rotation knob 5.

For this reason, applying the disclosure herein to the above-described configuration makes it possible to preferably realize an effect that it is possible to improve operability while inhibiting the conductive wire CA from breaking.

### Other Embodiments

The mode for carrying out the disclosure has been described and the disclosure should not be limited to only the above-described embodiment.

In the above-described embodiment, the conductive wire CA is electrically connected directly to the end effector 10 (the heater and the electrodes in a pair described above) via the sheath 6; however, the configuration is not limited thereto. A configuration in which the conductive wire CA is not arranged in the sheath 6 may be employed as long as the conductive wire CA is electrically connected to the end effector 10 via another member, such as the sheath 6.

In the above-described embodiment, the conductive wire CA is wound around the outer circumferential surface of the holder member body 762; however, the configuration is not limited thereto, and a configuration in which the conductive wire CA is wound around an outer circumferential surface of the fitting part 761 having a diameter smaller than that of the holder member body 762 may be employed.

Although the above-described embodiment does not refer to the number of conductive wires CA, a configuration in which multiple conductive wires CA are arranged may be employed.

In the above-described embodiment, the second operation portion according to the disclosure is not limited to the flex operation portion 9, and another operation portion may be employed as long as the operation portion is arranged on and end on the center axis Ax and on the proximal-end side Ar2 and that receives an operation of the user.

In the above-described embodiment, high-frequency energy and thermal energy are exemplified as the treatment energy that is applied to the subject region. Alternatively, an ultrasound energy, or the like, may be used. Here, “applying ultrasound energy to the subject region” means application of ultrasound vibrations to the subject region.

According to the treatment tool according to the disclosure, it is possible to improve operability.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
a sheath that extends along a first axis;
an end effector that is arranged on the sheath, the end effector being configured to apply energy to living tissue;
a first operation portion, the sheath being configured to be inserted into the first operation portion, the first operation portion being configured to rotate along the first axis together with the sheath;
a housing configured to support the first operation portion such that the first operation portion is configured to rotate along the first axis;
a conductive wire that extends into the housing and the first operation portion, is electrically connected to the end effector, and the conductive wire being a path configured to supply power to the end effector; and
a rotation portion that is at least partially positioned in the housing, the rotation portion being configured to rotate along the first axis together with the sheath and the first operation portion, and the conductive wire configured to wind around the rotation portion within the housing.

2. The treatment tool according to claim 1, wherein the end effector includes a pair of grippers that are configured to open and close,
the treatment tool further comprises:
a movable handle supported by the housing and configured to move according to an operation of the user; and
an elastic material that is arranged within the housing and that has an annular shape provided about the first axis, the elastic material being configured to apply a drive force to open and close the pair to the grippers, wherein:
the rotation portion is inserted into the elastic material.

3. The treatment tool according to claim 2, the rotation portion further comprising:
a first rotation part defining a tubular shape that extends along the first axis, the first rotation part being inserted into the elastic material, and
a second rotation part extending along the first axis and that is arranged in the first rotation part, wherein:
the conductive wire passes between the first rotation part and the second rotation part.

4. The treatment tool according to claim 3, wherein the rotation portion further includes a third rotation part that is positioned at a proximal end of the rotation portion and that is connected to at least one of the first rotation part and the second rotation part, and
the conductive wire is wound on an outer circumferential surface of the third rotation part along a circumferential direction about the first axis.

5. The treatment tool according to claim 4, wherein an annular concave groove that extends along the outer circumference surface, and
the conductive wire is wound in the concave groove.

6. The treatment tool according to claim 5, wherein a protrusion that is inserted into the concave is formed in the housing.

7. The treatment tool according to claim 1, wherein:
the end effector is configured to flex or curve relative to the sheath,
a proximal end part of the rotation portion is exposed to an outside of the housing, and
the treatment tool further comprises:
a second operation portion that is positioned on the first axis and that is supported by the proximal end part so as to be movable according to an operation of the user; and
a drive mechanism configured to cause the end effector to flex or curve with respect to the sheath according to move of the second operation portion.

8. The treatment tool according to claim 7, wherein the drive mechanism is arranged in the rotation portion.
